SEQUENCE LISTING (4 sequences)

SEQ ID NO: 1 - protein, 249 aa, HUMAN
QSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDNNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQ

SEQ ID NO: 2 - protein, 249 aa, HUMAN
MDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 3 - protein, 517 aa, HUMAN
MEWSWVFLFFLSVTTGVHSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDNNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 4 - protein, 498 aa, HUMAM
QSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

(19) United States
(12) Patent Application Publication
(10) Pub. No.: US 2015/0118228 A1
(43) Pub. Date: Apr. 30, 2015

(54) BROAD SPECTRUM ERBB LIGAND BINDING MOLECULES AND METHODS FOR THEIR USE

(71) Applicant: Ligacept LLC, Oak Brook, IL (US)

(72) Inventor: Jason E. Hill, Chicago, IL (US)

(21) Appl. No.: 14/396,965

(22) PCT Filed: Apr. 25, 2013
(86) PCT No.: PCT/US2013/038203

§ 371 Date: Oct. 24, 2014

Related U.S. Application Data
(60) Provisional application No. 61/638,312, filed on Apr. 25, 2012.

(51) Int. Cl. C12N 9/12; C07K 14/71

(52) U.S. Cl. CPC C12N 9/12; C07K 14/71; C12Y 207/10001; C07K 2319/70
USPC 424/134.1; 435/194; 536/23.2; 435/320.1; 435/361; 424/94.5; 435/328

(57) ABSTRACT

A chimeric ErbB ligand binding molecule is disclosed along with its pharmaceutically acceptable salt forms. The molecule is a protein that as part of its sequence includes the sequence of SEQ ID NOS: 1, 2, or 3. The molecule can be fused to an IgGFc and especially IgGFc containing cysteine to serine changes in the hinge region. For example, the fusion can be to IgG 1Fc DNA sequences that encode the binding molecules are also contemplated as well as vectors containing such DNA sequences and hosts that contain such vectors. Pharmaceutical compositions are contemplated that contain the binding molecule along with a pharmaceutically acceptable excipient.

### BACKGROUND

Receptor tyrosine kinases are involved in stimulating the growth of many cancers. In general, receptor tyrosine kinases are glycoproteins which consist of (1) an extracellular domain that can bind with a specific ligand, (2) a transmembrane region, (3) a juxtamembrane domain which may regulate the receptor activity by, for instance, protein phosphorylation, (4) a tyrosine kinase domain that is the enzymatic component of the receptor, and (5) a carboxyterminal tail. The ErbB family of type I receptor tyrosine kinases constitute an important class of such receptors because of their importance in mediating cell growth, differentiation and survival in many solid tumors. Members of this receptor family include ErbB1 (also known as HER1), ErbB2 (HER2/neu), ErbB3 (HER3), and ErbB4 (HER4). More than a dozen ligands interact with the ErbB-family receptors. For example, EGF, Transforming Growth Factor α (TGFα), and amphiregulin all bind to ErbB1. Isoforms of neuregulin, also known as Heregulin and Neu Differentiation Factor (NDF) have specific affinity for ErbB3 and ErbB4. Ligands such as betacellulin, heparin-binding EGF and epiregulin bind to both ErbB1 and ErbB4.

It is becoming clear that over expression of ErbB activating ligands can cause an uncontrolled cellular proliferation disease state similar to that of a deregulated receptor. In such cases, interference with the binding of the activating ligand to its receptor may provide an effective therapeutic strategy or could accentuate current receptor based or other therapies. Binding molecules that can trap and sequester the full spectrum of ErbB ligands may be of even more use in the treatment of cancer.

Several therapeutics exist that have attempted this trapping or “decoy” strategy. For example, Enbrel™ (etanercept—Amgen) is a soluble, modified version of the TNFR receptor that binds and traps the pro-inflammatory ligand TNFα. In addition, a soluble fusion protein of the VEGFRI and VEGFR2 receptors, called EYLEA™, has been approved for the treatment of macular degeneration and is under investigation for use in the treatment of several forms of cancer (Regeneron Pharmaceuticals). An ErbB3 trap has also shown potency in vitro at enhancing the effects of a dual EGFR/ErbB2 inhibitor and reversed GW2974 (a small molecule inhibitor of ErbB 1 and ErbB2) resistance in cells treated with NDF.

All currently approved ErbB inhibitors target either EGFR, ErbB2, ErbB3, ErbB4 or combinations of all 4 proteins. However, no therapeutic is known that interferes with the binding of ligands to multiple ErbB receptors simultaneously. Thus, new binding molecules are needed that can be used to sequester receptor ligands, such as ErbB ligands, and thereby block ligand binding to multiple ErbB receptors and subsequent receptor activation. Binding molecules capable of binding ligands to more than ErbB 1 or ErbB4 receptors and ideally all known ErbB ligands and that are accompanied by a minimal immune response would be particularly useful.

A number of binding studies have been carried out to determine regions of ErbB3 that are important to the binding of its ligand, heregulin. Singer, et. al. (2001), J. Biol. Chem. 276, 44266-44274. Other studies using chimeric receptors have identified the relative contributions of the extracellular domains of ErbB 1 and ErbB4 to ligand-specific signaling. Kim, et. al. (2002), Eur. J. Biochem. 269, 2323-2329. These studies reveal that neuregulin binding to ErbB4 depends much more on domain I than on domain III and that domain III of ErbB 1 is primarily important for EGF binding. However, these studies were conducted on full length receptors which span the entire length of the receptors including the transmembrane and cytoplasmic domains. These large molecules present manufacturing and administration problems potentially leading to lower therapeutic efficacy.

### SUMMARY OF INVENTION

A chimeric ErbB ligand binding molecule is disclosed along with its pharmaceutically acceptable salt forms. The molecule is a protein that as part of its sequence includes the sequence of SEQ ID NOS: 1, 2, or 3. The molecule can be fused to an IgGFc and especially IgGFc containing cysteine to serine changes in the hinge region. For example, the fusion can be to IgG1Fc DNA sequences that encode the binding molecules are also contemplated as well as vectors containing such DNA sequences and hosts that contain such vectors. Pharmaceutical compositions are contemplated that contain the binding molecule along with a pharmaceutically acceptable excipient.

Methods for treating a patient having a cancer that is sensitive to one or more ErbB ligands are also contemplated. Such methods can involve administering a therapeutically effective amount of a pharmaceutical composition that contains the chimeric ErbB ligand binding molecule.

### FIGURES

FIG. 1 provides a graphical representation of the decrease in p-EGFR expression as the concentration of LC010 increases.

FIG. 2 provides a graphical representation of the inhibition of p-EGFR in which the IC₅₀ was calculated.

FIG. 3 provides a graphical analysis of the decrease in p-ErbB3 expression of McF7 cells when treated with HRG1B in response to increasing LC010.

FIG. 4 provides a graphical analysis of the inhibition shown in FIG. 3 in which the IC₅₀ was calculated.

FIG. 5 provides a graphical analysis of the inhibition shown in FIG. 4 with the inhibition by Trap 6 in an identical experiment.

### DETAILED DESCRIPTION OF INVENTION

For purposes of this disclosure the term “homology” is intended to mean a region of amino acid sequence having identical or conservative amino acid substitutions as that term is generally understood in the art.

All of the amino acid numbering in this application is intended to be exclusive of the native signal peptide.

A chimeric ErbB embodiment has been developed in which amino acids 1-249 from ErbB4, SEQ ID NO. 1 below, are fused to amino acids 253-501 from ErbB1, SEQ ID NO. 2 below, as shown in SEQ ID NO.: 3. The fusion points described above for ErbB4 and ErbB1 were specifically chosen to reduce the immunogenicity of the chimeric protein. Glutamine 126 in the ErbB4 sequence can be changed to an asparagine to increase the binding affinity for ErbB 1 specific ligands.

In certain embodiments the ErbB chimera can be fused with components that cause aggregative conjugate formation or to extend protein half-life. For example, the ErbB chimera can be fused to the constant region of immunoglobulin molecule such as the Fc region of IgG. For purposes of this disclosure one suitable Fc region is IgG2Fc. Another is IgG1Fc. Others are also known in the art and can be used. In certain embodiments the moiety can contain mutations that reduce the tendency of the Fc to dimerize. This can include substitutions of serine in place of cysteine at positions 226 and 229 of the IgG2Fc moiety, for example.

SEQ. ID. NO. 1
Q S V C A G T E N K L S S L S D L E Q Q Y R A L R K Y Y E N C E V V M G N L E I T S I
E H N R D L S F L R S V R E V T G Y V L V A L N Q F R Y L P L E N L R I I R G T K L Y
E D R Y A L A I F L N Y R K D G N F G L Q E L G L K N L T E I L N G G V Y V D N N K F
L C Y A D T I H W Q D I V R N P W P S N L T L V S T N G S S G C G R C H K S C T G R C
W G P T E N H C Q T L T R T V C A E Q C D G R C Y G P Y V S D C C H R E C A G G C S
G P K D T D C F A C M N F N D S G A C V T Q C P Q T F V Y N P T T F Q
SEQ. ID. NO. 2
M D V N P E G K Y S F G A T C V K K C P R N Y V V T D H G S C V R A C G A D S Y E
M E E D G V R K C K K C E G P C R K V C N G I G I G E F K D S L S I N A T N I K H F K
N C T S I S G D L H I L P V A F R G D S F T H T P P L D P Q E L D I L K T V K E I T G F L
L I Q A W P E N R T D L H A F E N L E I I R G R T K Q H G Q F S L A V V S L N I T S L G
L R S L K E I S D G D V I I S G N K N L C Y A N T I N W K K L F G T S G Q K T K I I S N
R G E N S C K A T G Q V C H A L C S P E G C W G P E P R D C V S

The sequence of the chimera with a signal sequence (M E W S W V F L F F L S V T T G V H S) included is set out below:

SEQ ID NO. 3
M E W S W V F L F F L S V T T G V H S Q S V C A G T E N K L S S L S D L E Q Q Y R A
L R K Y Y E N C E V V M G N L E I T S I E H N R D L S F L R S V R E V T G Y V L V A L
N Q F R Y L P L E N L R I I R G T K L Y E D R Y A L A I F L N Y R K D G N F G L Q E L
G L K N L T E I L N G G V Y V D N N K F L C Y A D T I H W Q D I V R N P W P S N L T L
V S T N G S S G C G R C H K S C T G R C W G P T E N H C Q T L T R T V C A E Q C D G
R C Y G P Y V S D C C H R E C A G G C S G P K D T D C F A C M N F N D S G A C V T Q
C P Q T F V Y N P T T F Q M D V N P E G K Y S F G A T C V K K C P R N Y V V T D H G
S C V R A C G A D S Y E M E E D G V R K C K K C E G P C R K V C N G I G I G E F K D
S L S I N A T N I K H F K N C T S I S G D L H I L P V A F R G D S F T H T P P L D P Q E L
D I L K T V K E I T G F L L I Q A W P E N R T D L H A F E N L E I I R G R T K Q H G Q F
S L A V V S L N I T S L G L R S L K E I S D G D V I I S G N K N L C Y A N T I N W K K L
F G T S G Q K T K I I S N R G E N S C K A T G Q V C H A L C S P E G C W G P E P R D C
V S

Trap 6 is identical to LC010 with the exception of an Asparagine at position 126. Its sequence is provided with SEQ is No. 4 below:

SEQ ID NO. 4
Q S V C A G T E N K L S S L S D L E Q Q Y R A L R K Y Y E N C E V V M G N L E I T S I
E H N R D L S F L R S V R E V T G Y V L V A L N Q F R Y L P L E N L R I I R G T K L Y
E D R Y A L A I F L N Y R K D G N F G L Q E L G L K N L T E I L N G G V Y V D Q N K F
L C Y A D T I H W Q D I V R N P W P S N L T L V S T N G S S G C G R C H K S C T G R C
W G P T E N H C Q T L T R T V C A E Q C D G R C Y G P Y V S D C C H R E C A G G C S
G P K D T D C F A C M N F N D S G A C V T Q C P Q T F V Y N P T T Y Q M D V N P E G
K Y S F G A T C V K K C P R N Y V V T D H G S C V R A C G A D S Y E M E E D G V R K
C K K C E G P C R K V C N G I G I G E F K D S L S I N A T N I K H F K N C T S I S G D L
H I L P V A F R G D S F T H T P P L D P Q E L D I L K T V K E I T G F L L I Q A W P E N
R T D L H A F E N L E I I R G R T K Q H G Q F S L A V V S L N I T S L G L R S L K E I S
D G D V I I S G N K N L C Y A N T I N W K K L F G T S G Q K T K I I S N R G E N S C K
A T G Q V C H A L C S P E G C W G P E P R D C V S

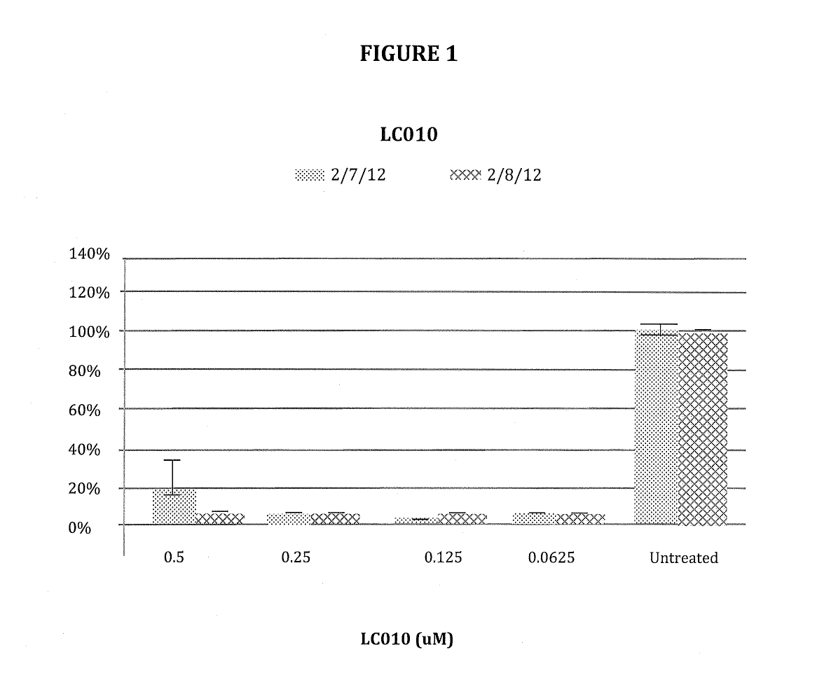

The chimeric ErbB binding molecule of SEQ ID NO.: 3 was fused to IgG2Fc having serine in place of cysteine at positions 226 and 229 on the Fc moiety (LC010) and the resulting protein was isolated. The ability of the molecule to inhibit the growth of A431 cells in the presence of added TGFα was investigated and is shown. Briefly, A431 cells were either treated with different concentrations of LC010 (62.5 to 500 nM) for 2 hrs or untreated. After 2 hrs, the cells were treated with 12.5 ng/mL of TGFα for 10 min Cell lysates were collected and analyzed for EGFR activation with a p-EGFR ELISA assay. The graph in FIG. 1 shows the decrease in p-EGFR expression with increasing concentration of LC010. Results of 2 independent experiments are shown in FIG. 1.

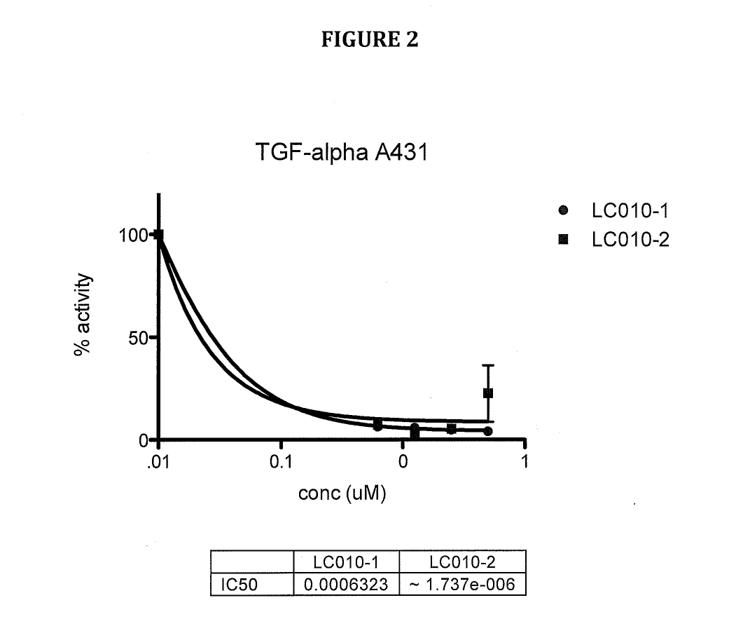

The IC₅₀ of LC010 inhibition of p-EGFR was plotted and is shown in FIG. 2. The IC₅₀ for LC010 in two experiments was calculated to be 0.6323 nM and 0.00173 nM.

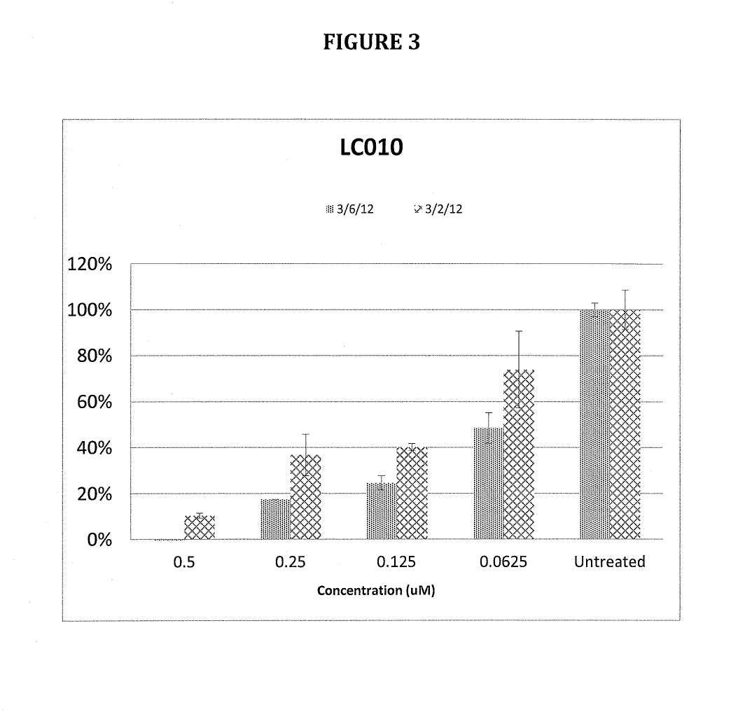

The chimeric ErbB binding molecule of SEQ ID NO.: 3 was fused to IgG2Fc having serine in place of cysteine at positions 226 and 229 on the Fc moiety (LC010) and the resulting protein was isolated. The ability of the molecule to inhibit the growth of MCF7 cells in the presence of added HRG1β was investigated and is shown in FIG. 3. Briefly, MCF7 cells were either treated with different concentrations of LC010 (62.5 to 500 nM) for 1 hr or untreated. After 1 hour the cells were treated with 12.5 ng/mL of HRG1β for 10 min. Cell lysates were collected and analyzed for ErbB3 activation with a p-ErbB3 ELISA assay. The graph in FIG. 3 shows the decrease in p-ErbB3 expression with increasing concentration of LC010. Results of 2 independent experiments are shown.

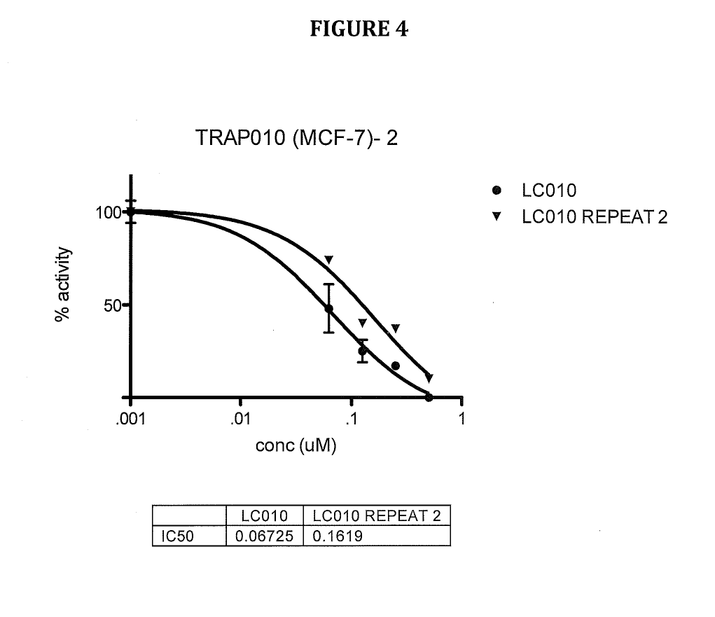

The IC₅₀ of LC010 inhibition of p-ErbB3 was plotted and shown in FIG. 4. The IC₅₀ for LC010 is in the nanomolar range. In two experiments the IC₅₀ was calculated to be 0.06725 and 0.1619 nM.

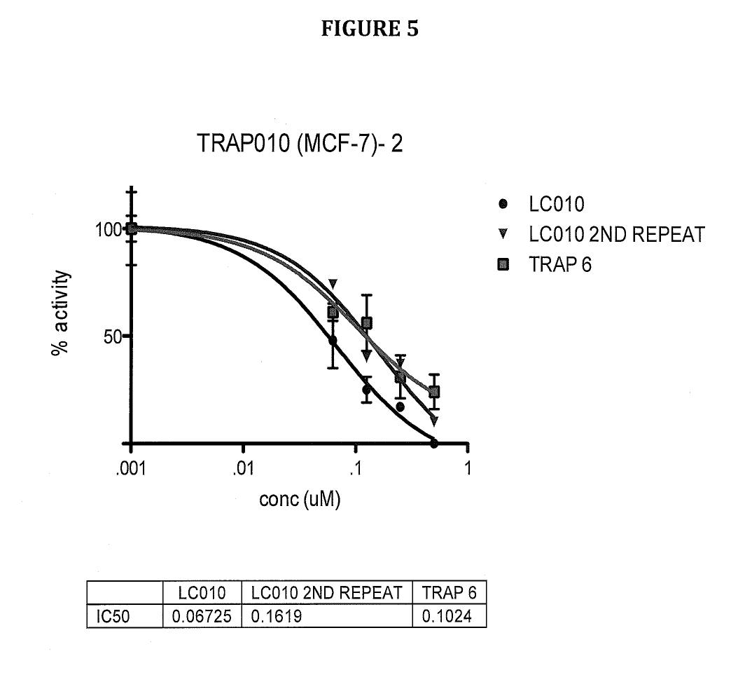

The IC₅₀ of LC010 inhibition of p-ErbB3 was plotted along with the IC₅₀ of Trap 6 (LC006) inhibition of p-ErbB3 for comparison and shown in FIG. 5.

Substitutions can be introduced into the amino acid sequence for a variety of purposes. For example, the DNA sequence for the chimeric binding molecule can be changed to remove cysteines so that the formation of aggregates through cysteine-cysteine bonds can be avoided. Substitutions of amino acids in one subdomain can be used to modify ligand binding affinities. For example, an amino acid from ErbB1 can be substituted into the ErbB4 LI subdomain to make that domain's sequence more like that of ErbB 1 in order to modify the affinity of the molecule to ErbB ligands. Similarly, amino acid substitutions from ErbB4 LII subdomains can be included into the ErbB 1 subdomain. Such substitutions can also be made in the SI and SII subdomains. Although any number of such substitutions can be considered substitutions of glutamine from ErbB 1 for serine in the ErbB4 portion at position 13, tyrosine for serine at position 42, arginine for tyrosine at position 123 are representative examples. Other examples can be identified by one of skill in the art simply by comparing sequences. Substitutions that are not homologous can also be considered. For example, asparagine could be substituted for serine at position 13 rather than the glutamine found in ErbB 1 or a residue that has intermediate characteristics of the residues found in both receptors may be used.

In addition to the Fe portion of IgG2, the chimeric ErbB protein could also be fused to other molecules or portions thereof including: other chimeric receptors (of any growth factor receptor family) or to sequences that facilitate purification of the product. The DNA sequences encoding the fusion proteins can be obtained from commercial sources and placed in any suitable expression vector and expressed from suitable hosts of which many are known.

DNA that encodes the chimeric ErbB ligand binding molecule sequences is also contemplated. One of skill can appreciate that the genetic code can be used to prepare suitable DNA sequences and codon preferences for specific expression hosts can also be incorporated into such sequences. Also contemplated for use with these sequences are additional DNA sequences that can be used for the expression of these DNA sequences. A variety of these are known. As is well known in the art such sequences can also be introduced into host cells for the maintenance of the DNA and for its expression and such hosts that include these DNA sequences are also contemplated.

Pharmaceutical compositions comprising a disclosed chimeric ErbB ligand binding molecule are also contemplated. Such compositions comprise a therapeutically effective amount of a chimeric ErbB ligand binding molecule, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle in which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like in which the chimeric ErbB ligand binding molecule is soluble and is chemically stable. The composition can also contain wetting or emulsifying agents, or pH buffering agents. These compositions can take the form of solutions, suspensions, emulsion, tablets, pills, capsules, powders, sustained-release formulations and the like. Pharmaceutically acceptable carriers include other ingredients for use in formulations such as DPPC, DOPE, DSPC and DOPC. Natural or synthetic surfactants may be used. PEG may be used (even apart from its use in derivatizing the protein or analog). Dextrans, such as cyclodextran, may be used. Cellulose and cellulose derivatives may be used. Amino acids may be used, such as use in a buffered formulation. Pharmaceutically acceptable diluents include buffers having various contents (e.g., Tris-HCl, acetate, phosphate), pH and ionic strength; additives such as detergents and solubilizing agents (e.g., Polysorbate 80), anti-oxidants (e.g., ascorbic acid, sodium metabisulfite), preservatives (e.g., benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form Implantable sustained release formulations are also contemplated, as are transdermal formulations. Liposome, microcapsule or microsphere, inclusion complexes, or other types of carriers are also contemplated.

The amount of the active chimeric binding molecule that will be effective for its intended therapeutic use can be determined by standard clinical techniques. In addition, in vitro assays may optionally be employed to help identify optimal dosage ranges. Generally, the daily regimen should be in the range of 0.1-1000 micrograms of the active agent (API) kilogram of body weight, preferably 0.1-150 micrograms per kilogram. Effective doses may be extrapolated from dose-response curves derived from in vitro or suitable animal model test systems. Dosage amount and interval may be adjusted individually to provide plasma levels of the compounds that are sufficient to maintain therapeutic effect. In cases of local administration or selective uptake, the effective local concentration of the compounds may not be related to plasma concentration. The dosage regimen involved in a method for treatment can be determined by the attending physician, considering various factors which modify the action of drugs, e.g. the age, condition, body weight, sex and diet of the patient, the severity of disease, time of administration and other clinical factors.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

The fusion protein of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Furthermore, aqueous compositions useful for practicing the methods of the invention have physiologically compatible pH and osmolality. One or more acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in an acceptable range. One or more acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

The amount of the fusion protein that will be effective for its intended therapeutic use can be determined by standard clinical techniques based on the present description. In addition, in vitro assays may optionally be employed to help identify optimal dosage ranges. Generally, suitable dosage ranges for intravenous administration are generally about 50-5000 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

A method for treating a patient in need of treatment is disclosed that includes obtaining a chimeric ErbB ligand binding molecule that binds ErbB ligands and interferes with the interaction and effect of ligands on the ErbB receptor system of cancer cells, and administering a therapeutically effective amount of the molecule to a patient. Administration can be by parenteral routes such as i.v. administration, direct injection into a solid tumor such as through a syringe or catheter or by i.p. injection.

In one method of treatment the chimeric ErbB ligand binding molecules can be immobilized to a solid support such as an apheresis or biocore support by standard methods. When the binding molecule is immobilized to a solid support the serum, blood or other biologically relevant fluid of a patient can be placed in contact with the solid support in the apheresis column to remove ErbB ligands from the fluid. The serum, blood or fluid can then be reintroduced into the patient.

The binding molecules can also be used in combination therapies. Thus, the chimeric ErbB ligand binding molecule may be administered in combination with one or more additional compounds or therapies, including chemotherapeutic agents, surgery, catheter devices, and radiation. Combination therapy includes administration of a single pharmaceutical dosage formulation which contains a chimeric ErbB ligand binding molecule and one or more additional agents. The chimeric ErbB ligand binding molecule and one or more additional agent(s) can be administered in their own separate pharmaceutical dosage formulations or together in the same formulation. For example, a chimeric ErbB ligand binding molecule and a cytotoxic agent, a chemotherapeutic agent or a growth inhibitory agent can be administered to the patient together in a single dosage composition or each agent can be administered in a separate dosage formulation. More specifically, the chimeric ErbB ligand binding molecule can be used in combination therapies that include therapeutic agents such as Lapatinib®, Herceptin®, Erbitux® and the like. Where separate dosage formulations are used, the chimeric ErbB ligand binding molecules and one or more additional agents can be administered concurrently, or at separately staggered times, i.e., sequentially. One of skill in the art can appreciate that the combination must be such that the chimeric ErbB ligand binding molecule does not interfere, but rather, accentuates the second therapeutic in the combination.

The invention also provides an article of manufacturing comprising packaging material and a pharmaceutical agent contained within the packaging material, wherein the pharmaceutical agent comprises at least one ErbB-binding fusion protein of the invention, and wherein the packaging material comprises a label or package insert which indicates that the ErbB-specific fusion protein can be used for treating an ErbB-mediated disease or condition.

Nucleotide sequences that encode the disclosed amino acid sequences are also contemplated. In addition, the conservative replacement of an amino acid with another similar amino acid that does not substantially (about 10-fold) interfere with ligand binding activity is specifically contemplated.

The DNA molecule was synthesized by starting with the desired amino acid sequence and optimizing the DNA sequence for mammalian system expression. The sequence was cloned into a suitable mammalian expression vector (pCpGfree-vitroHmcs) that can be selected using hygromycin and contains MAR/SAR sequences (insulator and boundary regions) and promoters and enhancers for trap expression. Vectors containing the sequence were transfected into CHO cells by standard transfection methods and the cells were selected for vector integration with hygromycin. Traps were purified from stably transfected cell lines by collecting cell culture medium and purifying by standard methods (protein A column binding). The chimeric protein was eluted from protein A by standard methods and quantitated using a custom derived ErbB 1 capture and IgG-Fc detection sandwich ELISA assay.

1. A chimeric ErbB ligand binding molecule and its pharmaceutically acceptable salt forms comprising SEQ ID NOS: 1, 2, or 3.

2. The chimeric ErbB ligand binding molecule of claim 1 and its pharmaceutically acceptable salt forms comprising SEQ ID NO: 3.

3. The chimeric ErbB ligand binding molecule of claim 1 and its pharmaceutically acceptable salt forms wherein the ErbB ligand binding molecule is fused to a portion of an IgGFc.

4. The chimeric ErbB ligand binding molecule of claim 1 and its pharmaceutically acceptable salt forms wherein the ErbB ligand binding molecule is fused to a portion of an IgGFc wherein the IgGFc is IgG1Fc.

5. A DNA sequence encoding a chimeric ErbB ligand binding molecule comprising SEQ ID NOS: 1, 2, or 3.

6. The DNA sequence of claim 4 further comprising an additional DNA sequence for expressing the chimeric ErbB ligand binding molecule in a host.

7. The DNA sequence of claim 4 wherein the DNA sequence is in a living host cell.

8. A pharmaceutical composition comprising a chimeric ErbB ligand binding molecule and its pharmaceutically acceptable salt forms comprising SEQ ID NOS: 1, 2, or 3 and a pharmaceutically acceptable excipient.

9. The pharmaceutical composition of claim 7 comprising a chimeric ErbB ligand binding molecule and its pharmaceutically acceptable salt forms comprising SEQ ID NO: 3 and a pharmaceutically acceptable excipient.

10. A method for treating a patient having a cancer that is sensitive to one or more ErbB ligands comprising administering a therapeutically effective amount of a pharmaceutical composition comprising a chimeric ErbB ligand binding molecule and its pharmaceutically acceptable salt forms comprising SEQ ID NOS: 1, 2, or 3 and a pharmaceutically acceptable excipient.

11. The method for treating a patient having a cancer that is sensitive to one or more ErbB ligands of claim 9 comprising administering a therapeutically effective amount of a pharmaceutical composition comprising a chimeric ErbB ligand binding molecule and its pharmaceutically acceptable salt forms comprising SEQ ID NO: 3 and a pharmaceutically acceptable excipient.
